(19) United States
(12) Patent Application Publication
(10) Pub. No.: US 2005/0049537 A1
(43) Pub. Date: Mar. 3, 2005

(54) Flexible wrap for supporting a portion of a body

(75) Inventors: Ricky W. Purcell, Alpharetta, GA (US); Laura F. Braunecker, Alpharetta, GA (US)

Correspondence Address: Schwegman, Lundberg, Woessner & Kluth, P.A., P.O. Box 2938, Minneapolis, MN, 55402, US

(73) Assignee: Kimberly-Clark Worldwide, Inc.

(21) Appl. No.: 10/648,589

(22) Filed: Aug. 25, 2003

(51) Int. Cl. A61L 15/00

(52) U.S. Cl. 602/75

(57) ABSTRACT

A flexible wrap and method for supporting a portion of a body. The flexible wrap includes an elastic band and an adhesive mounted on the elastic band near an end of the elastic band. The adhesive secures the elastic band to a body and is adapted to be detachably mounted to the body. In another form, the flexible wrap includes an elastic band and a plurality of fingers that extend from an end of the elastic band. Each of the plurality of fingers is secured to an exposed section of the elastic band when the flexible wrap is attached to the body. In still another form, the flexible wrap includes an elastic band and a pack having a midsection. The flexible wrap further includes a fastener, such as an adhesive, that extends along the midsection of the pack and secures the pack to the elastic band.

### FIELD

This invention relates in general to a flexible wrap, and in particular to a flexible wrap that supports various parts of a body.

### BACKGROUND

Applying heat or cold to various parts of a body is a recognized practice for curing or alleviating multiple kinds of physical problems. As an example, ice packs are often applied in an effort to reduce swelling associated with bruises or various types of surgery. In addition, heat is sometimes applied to various portions of the body to alleviate discomfort caused by injury (e.g., muscular strain).

Some of the conventional devices which are used to apply heat or cold to the body include heating pads and ice bags. One concern with using heating pads and ice bags is that they are difficult to attach to an injured area of a body because they typically need to be manually held in place by the injured person or someone assisting the injured person.

One method of maintaining hot or cold packs in place against an injured area of the body includes placing a pack against the body and wrapping a towel several times around the body such that the pack is sandwiched between the injured area and the towel. The free end of the towel is then is attached to an exposed and already-wrapped section of the towel to prevent the towel from unwinding.

Wrapping towels around the body in this manner can be quite cumbersome. Furthermore, towels are seldom able to maintain a pack in its desired location, especially when a patient moves around. In addition, when a towel is wrapped more tightly around the body in an attempt to help keep the ice or cold pack in place, the towel may cause patient discomfort.

Another method of treating an injured area of a body includes using a flexible wrap to support the injured area of the body. Flexible wraps may also be used to hold hot or cold packs against the injured area of the body.

One concern with using a flexible wrap is that it is usually difficult to mount flexible wraps on a body because at least a portion of the flexible wrap needs to be fixed relative to the body before the flexible wrap can be stretched around the body. Since there is no simple way to initially fix a flexible wrap relative to the body, wrapping a flexible wrap around an injured area on a body can be unwieldy.

Another concern with using flexible wraps is that they typically apply uneven pressure to the injured areas of the body that they are meant to treat/support. A flexible wrap applies uneven pressure because each portion of the body includes numerous contours. The contours on the body cause certain portions of the flexible wrap to stretch more or less than other portions as the flexible wrap is wrapped around the body. Applying uneven pressure to an injured area of the body can lead to increased patient discomfort and increased risk of further injury.

Some flexible wraps include pockets that are adapted to retain hot or cold packs. The packs are positioned against an injured portion of a body as the flexible wrap is wrapped around the body.

One drawback with including pockets in flexible wraps is that the pockets add unwanted expense to the cost of producing flexible wraps. A pocket also limits the size of the pack that may be inserted into the pocket. In addition, pockets often provide inadequate support to a pack, especially when the pocket is much bigger than the pack.

### SUMMARY OF THE INVENTION

The present invention relates to a flexible wrap for supporting a portion of a body. The flexible wrap is easily manufactured and can be quickly wrapped around an injured area on a body. The support provided by the flexible wrap can be customized over the width of the flexible wrap based on the contours of the body at that portion of the body where the flexible wrap is applied. The flexible wrap is also capable of holding a pack in place against an injured portion of the body.

In one aspect, the present invention relates to a flexible wrap for supporting a portion of a body. The flexible wrap includes an elastic band and an adhesive mounted on the elastic band near an end of the elastic band. The adhesive secures the elastic band to a body and is adapted to be detachably mounted to the body. The adhesive makes it easier for a user or therapist to affix the flexible wrap to the body so that the flexible wrap can be quickly wrapped around an injured portion of a body.

In another aspect, the present invention relates to a flexible wrap for supporting a portion of a body. The flexible wrap includes an elastic band and a plurality of fingers that extend from an end of the elastic band. Each of the plurality of fingers is secured to an exposed section of the elastic band when the flexible wrap is attached to the body. The plurality of fingers allows a user or therapist to customize the amount of pressure that is applied over the width of the flexible wrap to the injured area of the body.

In still another aspect, the present invention relates to a flexible wrap for supporting a portion of a body. The flexible wrap includes an elastic band and a pack having a midsection. The flexible wrap further includes a fastener, such as an adhesive, that extends along the midsection of the pack and secures the pack to the elastic band. The adhesive that extends along the midsection of the pack allows the pack to be secured to the elastic band without including a pocket on the elastic band.

In a sample form of the flexible wrap, the adhesive on the pack may be oriented transverse to the direction that the elastic band is stretched as the elastic band is wrapped around the body (i.e., transverse to the lateral edges of the elastic band). Orienting the adhesive transverse to the lateral edges of the elastic band minimizes the stress generated on the joint between the elastic band and the pack as the elastic band is stretched around the body.

In yet another aspect, the present invention relates to a method of supporting a portion of a body. The method includes attaching an elastic band to the body using an adhesive and wrapping the elastic band around the portion of the body. In some sample forms of the method, attaching an elastic band to the body using an adhesive includes attaching an end of the elastic band to skin on the body.

In an alternative aspect, the method of supporting a portion of a body includes wrapping an elastic band around the portion of the body and securing a plurality of fingers that project from an end of the elastic band to an exposed section of the elastic band. The method may further include repositioning at least one finger to customize pressure applied by the elastic band. As an example, repositioning at least one finger may include disengaging the at least one finger from the elastic band and then securing the at least one finger to another part of the elastic band.

The purposes and features of the present invention will be set forth in the description that follows. Additional features of the invention will be realized and attained by the product and processes particularly pointed out in the written description and claims hereof, as well as from the appended drawings.

It is to be understood that both the foregoing general description and the following detailed description are exemplary and are intended to provide further explanation of the invention claimed. The accompanying drawings, which are incorporated in and constitute part of this specification, are included to illustrate and provide a further understanding of the invention.

### BRIEF DESCRIPTION OF THE DRAWINGS

The present invention will be more fully understood, and further features will become apparent, when reference is made to the following detailed description and the accompanying drawings. The drawings are merely representative and are not intended to limit the scope of the claims. Like parts depicted in the drawings are referred to by the same reference numerals.

FIG. 1 illustrates a top view of a flexible wrap.

FIG. 2 illustrates a side view of the flexible wrap shown in FIG. 1.

FIG. 3 illustrates the flexible wrap of FIG. 1 as a first end of the flexible wrap is being applied to a body.

FIG. 4 illustrates the flexible wrap of FIG. 1 as the flexible wrap is being wrapped around a portion of the body.

FIG. 5 illustrates a top view of another flexible wrap.

FIG. 6 illustrates a section view of the flexible wrap shown in FIG. 5 taken along line 6-6.

FIG. 7 illustrates the flexible wrap of FIG. 5 after the flexible wrap has been applied to a body.

FIG. 8 illustrates the flexible wrap of FIG. 7 after fingers on the flexible wrap have been repositioned to modify the pressure applied by the flexible wrap on the body.

FIG. 9 illustrates a top view of still another flexible wrap.

FIG. 10 illustrates a top view of yet another flexible wrap.

FIG. 11 illustrates a pack used in the flexible wrap shown in FIG. 10.

FIG. 12 illustrates a top view of the flexible wrap shown in FIG. 10 with the flexible wrap in a stretched condition.

FIG. 13 illustrates the flexible wrap shown in FIG. 10 after the flexible wrap has been applied to a body and the pack shown in FIG. 11 is positioned near an injured portion of the body.

### DETAILED DESCRIPTION OF THE INVENTION

In the following detailed description, reference is made to the accompanying drawings, which show specific embodiments in which the invention may be practiced. These embodiments are described in sufficient detail to enable those skilled in the art to practice the invention. It is to be understood that other embodiments may be utilized and structural changes made, such that the following detailed description is not to be taken in a limiting sense.

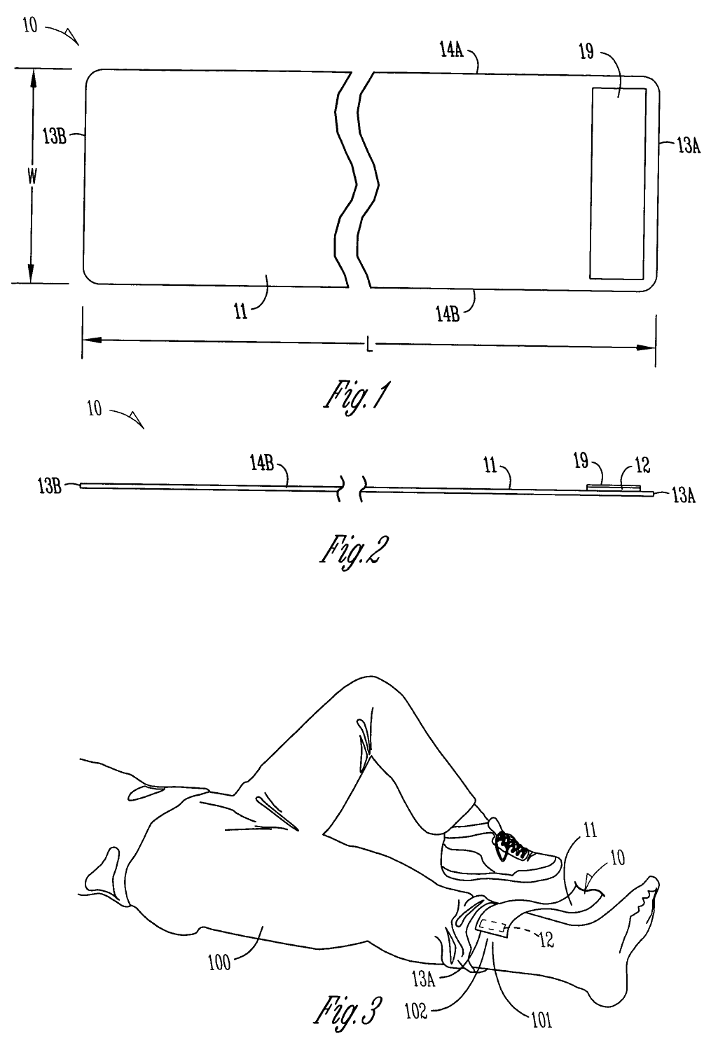

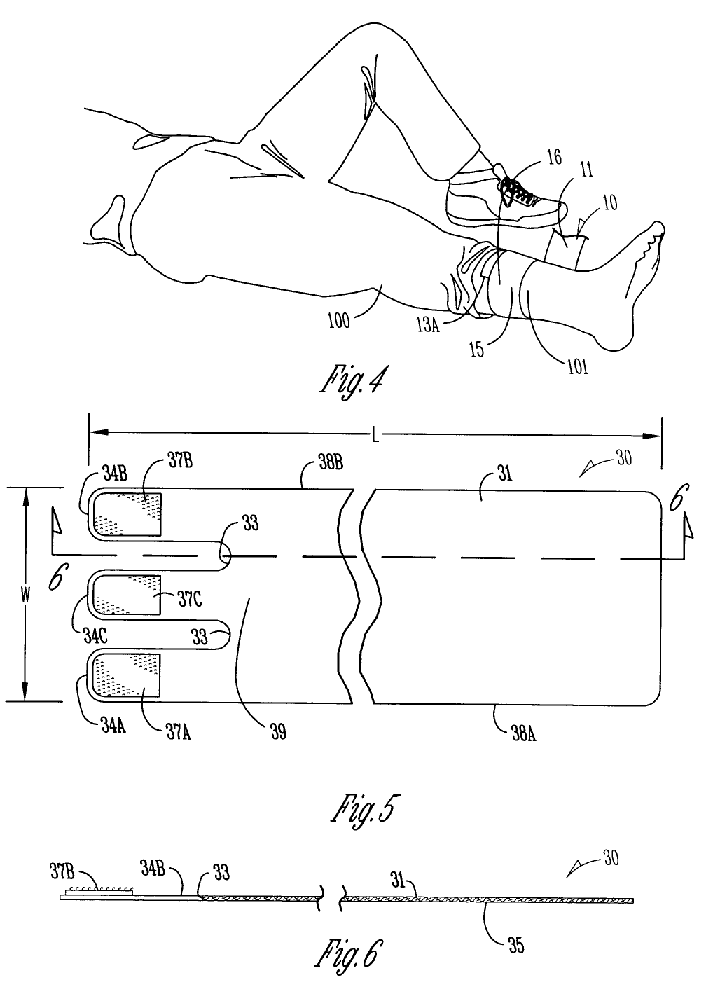

FIGS. 1-4 illustrate a flexible wrap 10 for supporting a portion 101 of a body 100. The flexible wrap 10 includes an elastic band 11 and an adhesive, such as adhesive layer 12 (shown in FIGS. 2-3), which is mounted on the elastic band 11 near an end 13A of the elastic band 11. The adhesive layer 12 is adapted to be detachably mounted to the body 100. In the sample flexible wrap 10 illustrated in FIGS. 1-4, adhesive layer 12 extends substantially between lateral edges 14A, 14B of the elastic band 11 near the end 13A of the elastic band 11. It should be noted that in some forms the adhesive layer 12 extends along the edge 13A of the elastic band 11.

The flexible wrap 10 may further include a cover 19 that is detachably mounted to the adhesive layer 12. The cover 19 is removed to expose the adhesive layer 12 so that the adhesive layer 12 can be used to attach the flexible wrap 10 to a desired location on the body 100.

The adhesive may be styrene-isoprene-styrene copolymer, silicone or hydrogel. Although any conventional adhesive may be used to attach the elastic band 11 to the body 100, some adhesives may be adapted to be detachably mounted directly to skin 102 on the body 100. Applying one end 13A of the elastic band 11 directly to the skin 102 on the body 100 simplifies the process of wrapping the flexible wrap 10 around the body 100.

The elastic band 11 may be formed from one or more of layers (only one layer is shown in FIGS. 1-4). The number and type of layers will depend on the application where the flexible wrap 10 is used. As an example, some layers may be more elastic while other layers may be made of softer and/or more insulating materials. The elastic band 11 may be made out of an elastomeric non-woven material, or a non-woven laminate, such as Vertical Filament Laminate or CFSBL. The elastic band 11 may be created by folding material in half and securing the edges of the material together using adhesives, or some other known fastening techniques.

The proper size and shape of the elastic band 11 will also depend on the application where the flexible wrap 10 is used. The appropriate length L and width W will be determined in part by the size and shape of the injured portion 101 on the body 100. In some forms, the elastic band 11 may be long enough to fit around the arms, legs, head or torso of a human being.

The flexible wrap 10 is applied to the body 100 by attaching the adhesive layer 12 to the body 100 at a location near the injured portion 101 of the body (FIG. 3). It should be noted that the adhesive layer 12 may be attached directly to skin 102 on the body 100 or to some form of covering (e.g., bandage, clothing) covering the body 100. Once the flexible wrap 10 is attached to the body 100, the elastic band 11 is wrapped around the injured portion 101 of the body 100 (FIG. 4). The elastic band 11 is wrapped around the body 100 until a second end 13B of the elastic band 11 is secured to an outer surface 15 of an already-wrapped and exposed section 16 of the elastic band 11. The second end 13B may be tucked into an already-wrapped section of the elastic band 11, or secured using any conventional type of fastener.

In some forms, the elastic band 11 may be treated with a variety of therapeutic additives, such as herbs, vitamins, drugs that are epidermal effective, and/or botanicals. Some example botanicals include lavender, chamomile, moisturizers, lipids, essential oils and fragrances. The therapeutic additives could be sprayed, or gravure printed, directly on the elastic band 11.

In alternative forms, the additives could be applied to a lightly absorbent pad (not shown) that is situated between layers of the elastic band 11. In some forms, the absorbent pad may be a stretchable absorbent foam.

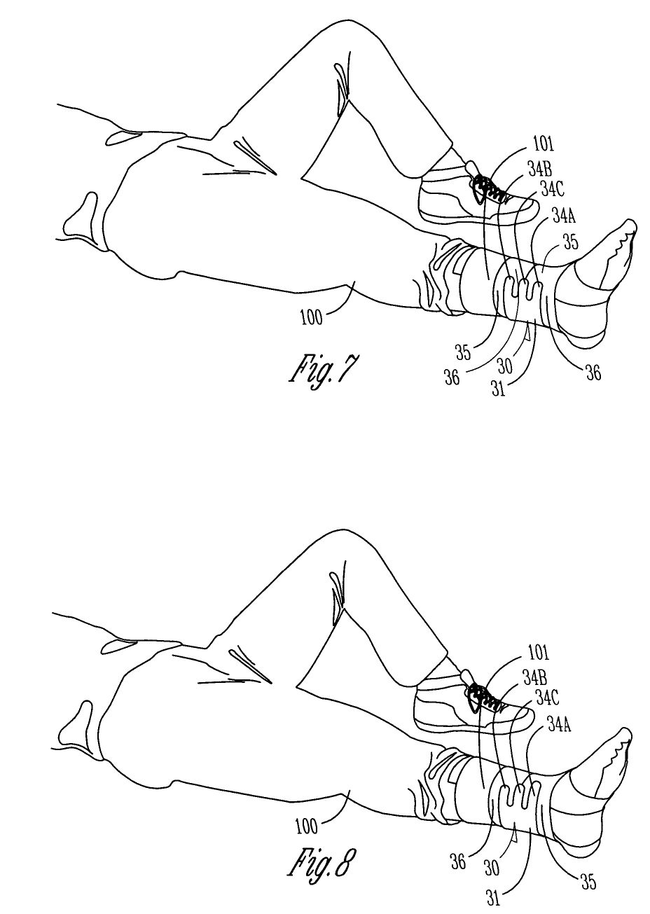

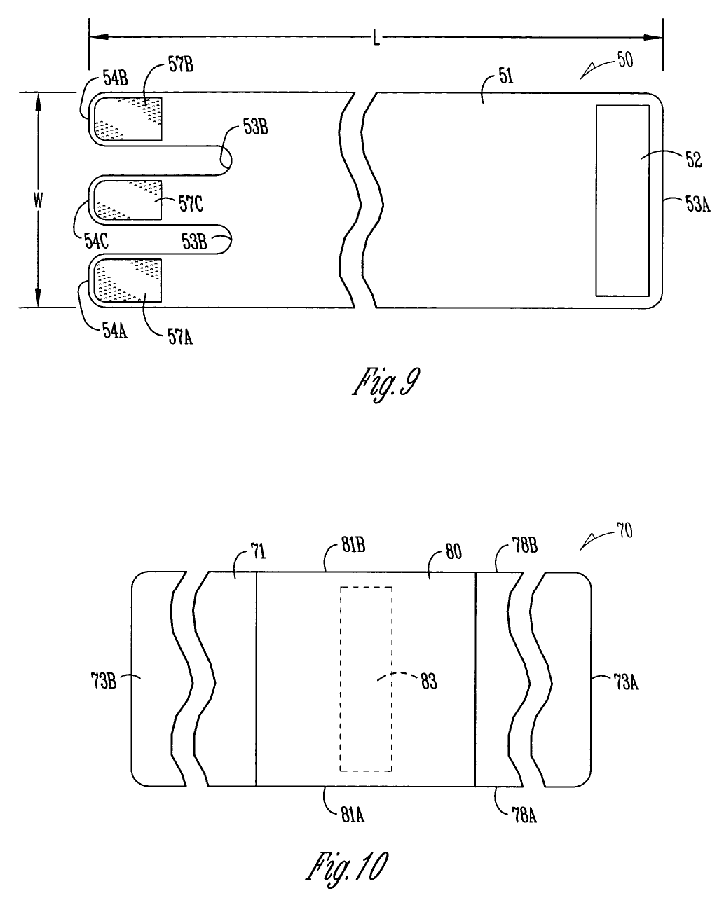

FIGS. 5-8 illustrate another flexible wrap 30 for supporting a portion 101 of a body 100. The flexible wrap 30 includes an elastic band 31 and a plurality of fingers 34A, 34B, 34C that extend from an end 33 of the elastic band 31. The plurality of fingers 34A, 34B, 34C are secured to an outer surface 35 of an already-wrapped and exposed section 36 on the elastic band 31 when the flexible wrap 30 is attached to the body 100 (see, e.g., FIGS. 7-8).

The flexible wrap 30 may further include a plurality of fasteners 37A, 37B, 37C such that each of the fingers 34A, 34B, 34C includes at least one of the fasteners 37A, 37B, 37C. The fasteners 37A, 37B, 37C are used to secure the plurality of fingers 34A, 34B, 34C to the exposed section 36 (shown in FIG. 7-8) of the elastic band 31. A hook and loop fastener, such as VELCRO, is one example type of fastener that may used when the elastic band 31 includes an outer layer which is formed from an appropriate material. It should be noted that any type of fastener or adhesive may used to secure the fingers 34A, 34B, 34C to the outer surface 35 of the elastic band 31.

In the sample embodiment illustrated in FIGS. 5-8, the fingers 34A, 34B, 34C are integral with the elastic band 31. In addition, one of the fingers 34A is aligned with one lateral edge 38A of the elastic band 31 while another of the fingers 34B is aligned with the other lateral edge 38B of the elastic band 31. The third finger 34C extends from a midsection 39 of the end 33 on the elastic band 31 (FIG. 5).

It should be noted that the number, size and arrangement of the fingers on the flexible wrap 30 may vary depending on the application where the flexible wrap 30 is being used. In addition, the fingers may be attached to any portion of the elastic band as long as the fingers extend beyond an end of the elastic band. In other sample forms of the flexible wrap, there may be fasteners on none, one, some or all of the fingers that extend from an end of the elastic band.

The flexible wrap 30 is applied to the body 100 in any conventional manner. The elastic band 31 is then wrapped around the injured portion 101 of the body 100 until the fingers 34A, 34B, 34C that extend from the end 33 of the elastic band 31 are secured to the outer surface 35 of the exposed section 36 on the elastic band 31 (FIG. 7).

The pressure applied by the flexible wrap 30 may be adjusted along the width W of the flexible wrap 30 by releasing one or more of the fingers 34A, 34B, 34C from the elastic band 31 and then reattaching the fingers 34A, 34B, 34C at a new location along the length L of the elastic band 31 (fingers 34A and 34C are adjusted in FIG. 8). Therefore, the fingers 34A, 34B, 34C allow a user or therapist to compensate for the contours of the body 101 by customizing the pressure applied along the width W of the flexible wrap 30. Although flexible wrap 30 is shown as being spiraled many times around the injured portion 101 of the body 100, it should be noted that flexible wrap 30 may be more effective when the elastic band 31 is wrapped around the body more than time but less than two times.

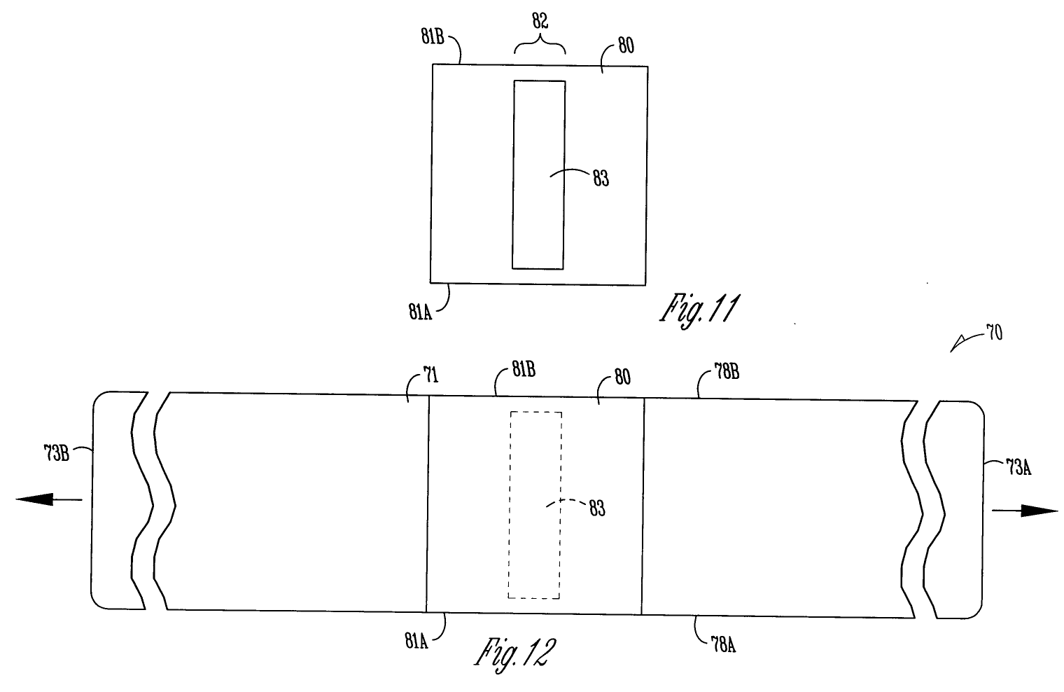

FIG. 9 illustrates another flexible wrap 50 for supporting a portion of a body. The flexible wrap 50 includes an elastic band 51 and an adhesive, such as adhesive layer 52, which is mounted on the elastic band 51. The adhesive layer 52 is mounted near an end 53A of the elastic band 51 and is adapted to be detachably mounted to the body.

The flexible wrap 50 further includes a plurality of fingers 54A, 54B, 54C and a plurality of fasteners 57A, 57B, 57C such that each finger 54A, 54B, 54C includes at least one of the fasteners 57A, 57B, 57C. The fingers 54A, 54B, 54C extend from a second end 53B of the elastic band 51. The pressure applied by the flexible wrap 50 may be adjusted along the width W of the flexible wrap 50 by repositioning the fasteners 57A, 57B, 57C on one or more of the fingers 54A, 54B, 54C along the length L of the flexible wrap 50.

As discussed above with regard to elastic band 11 in FIGS. 1-4, elastic band 51 may be formed from one or more of layers. The number and type of layers will depend on such factors as the type of application where the flexible wrap 50 is used and the type of fasteners 57A, 57B, 57C that are employed on the fingers 54A, 54B, 54C (among other factors).

FIGS. 10-13 illustrate all, or parts of, another flexible wrap 70 for supporting a portion 101 of a body 100. The flexible wrap 70 includes an elastic band 71 and a pack 80. Pack 80 may a hot pack, cold pack or any conventional type of pack or patch.

In some forms, pack 80 may be applied to an affected skin area to deliver treatment from various skin ailments. Some example ailments include irritation due to sun bums, heat bums, insect bites, athletes foot, dry skin, or other chronic skin conditions.

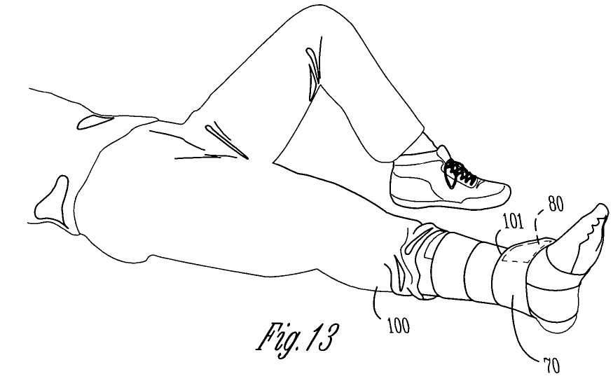

The illustrated example pack 80 includes a pair of opposing edges 81A, 81B and a midsection 82 (FIG. 11). The flexible wrap 70 further includes a fastener, such as adhesive layer 83, which extends along the midsection 82 of the pack 80. The adhesive layer 83 secures the pack 80 to the elastic band 71. In some forms, the pack 80 may include an adhesive (not shown) on the side of the pack 80 that is opposite to the elastic band 71, such that the pack could initially be placed against the skin with pressure before being further secured by the flexible wrap 70.

In the example flexible wrap 70 illustrated in FIGS. 10-13, the adhesive layer 83 extends substantially between the opposing edges 81A, 81B of the pack 80 (FIG. 11) and is transverse to the lateral edges 78A, 78B of the elastic band 71 when the pack 80 is secured to the elastic band 71 (FIGS. 10 and 12).

Although the adhesive layer 83 is shown as extending substantially between the lateral edges 78A, 78B of the elastic band 71 when the pack 80 is secured to the elastic band 71, it should be noted that the adhesive layer 83 may extend to one, both or none of the lateral edges 78A, 78B on the elastic band 71. In addition, the adhesive layer 83 may be formed of one or more sections (only one section is shown in FIGS. 10-13).

The flexible wrap 70 may further include a cover (not shown) that is detachably mounted to the adhesive layer 83. The cover is removed to expose the adhesive layer 83 so that the adhesive layer 83 can be used to mount the pack 80 to a desired location on the elastic band 71.

The flexible wrap 70 is applied to the body 100 by securing the pack 80 to the elastic band 71 such that the adhesive layer 83 is transverse to the lateral edges 78A, 78B of the elastic band 71. The flexible wrap 70 is then secured relative to the body 100 at a location near the injured portion 101 of the body 100. Once the flexible wrap 70 is secured relative to the body 100, the elastic band 71 is wrapped around the body 100 such that the pack 80 is near the injured portion 101 of the body 100 (FIG. 13). The elastic band 71 is wrapped around the body 100 until one of the ends 73A, 73B of the elastic band 71 is secured to an outer surface of an already-wrapped section of the elastic band 71.

The pack 80 may also be applied to the elastic band 71 after a portion of the elastic band 71 has been wrapped around the body 100. The pack 80 may be applied anywhere along the length L of the elastic band 71 as long as the pack 80 is positioned near the injured portion 101 of the body 100.

The flexible wrap 70 is shown in FIG. 10 in a relaxed condition and in FIG. 12 in a stretched condition. Positioning the adhesive layer 83 on the midsection 82 of the pack 80, and orienting the adhesive layer 83 transverse to the lateral edges 78A, 78B of the elastic band 71, minimizes the stress that is generated on the joint between the elastic band 71 and the adhesive layer 83 when the elastic band 71 is stretched as part of being wrapped around the body 100. The location and orientation of the adhesive layer 83 relative to the elastic band 71 and the pack 80 allows the pack 80 to be reliably secured to the elastic band 71 without using a pocket formed on the elastic band 71.

In some forms, the side of the pack 80 that is not attached to the elastic band 71 may include lotion that gets applied to the skin. The flexible wrap 70 serves to prevent the lotion from being removed by clothes, sheets, or furniture. The flexible wrap 70 also helps to inhibit the lotion from drying out such that lotion would have to be applied less frequently. The need to reapply the lotion may be reduced even further when a reserve of lotion (or some other material) is stored within the pack 80. Reducing the need to reapply lotion, or some other material, minimizes the pain associated with changing garments.

A method of supporting a portion 101 of a body 100 is described herein with reference to FIGS. 1-4. The method includes attaching an elastic band 11 to the body 100 using an adhesive, such as adhesive layer 12. The method further includes wrapping the elastic band 71 around the portion 101 of the body 100.

The method may further include (i) securing an end 13B of the elastic band 11 to an outer surface 15 of an already-wrapped and exposed section 16 on the elastic band 11; and/or (ii) exposing the adhesive layer 12 by removing a cover 19 from the adhesive layer 12. In some sample forms of the method, attaching the elastic band 11 to the body 100 includes attaching an end 13A of the elastic band 11 to the body 100, and/or attaching the adhesive layer 12 to skin 102 on the body 100.

Another form of the method of supporting a portion 101 of a body 100 is described herein with reference to FIGS. 5-8. The method includes wrapping an elastic band 31 around the portion 101 of the body 100 and securing a plurality of fingers 34A, 34B, 34C that project from an end 33 of the elastic band 31 to an exposed section 36 of the elastic band 31.

The method may further include repositioning at least one of the fingers 34A, 34B, 34C to customize pressure applied by the elastic band 31 to the portion 101 of the body 100. In some sample forms of the method, repositioning at least of the one fingers 34A, 34B, 34C includes disengaging at least one of the fingers 34A, 34B, 34C from the elastic band 31 and then securing the finger(s) 34A, 34B, 34C to another part of the elastic band 31 (see FIG. 8).

The method may further include attaching the elastic band 31 to the body 100. As an example, an adhesive may be used to attach the elastic band 31 directly to skin 102 on the body 100 (not shown in FIGS. 5-8, but see, e.g., flexible wrap 10 in FIG. 3).

The operations discussed above with respect to the described methods may be performed in a different order from those described herein. It should be noted that attaching a flexible wrap to a body includes attaching the flexible wrap directly or indirectly to the body. In addition, FIGS. 1-13 are representational and are not necessarily drawn to scale. Certain proportions thereof may be exaggerated, while others may be minimized.

The flexible wraps and methods described herein allow a user or therapist to customize the amount of pressure that is applied over the width of the flexible wrap to an injured area of a body. The flexible wraps and methods are also easily manufactured and readily affixed to the body so that the flexible wrap can be quickly wrapped around an injured portion of a body. In some forms, the flexible wrap holds a pack in place against an injured portion on the body without the use of a pocket formed on the flexible wrap. Some example applications include wearing the flexible wrap to relieve pain, treat afflictions, promote weight-loss, and/or for relaxation to facilitate sleep.

While the invention has been described in detail with respect to the specific aspects thereof, it will be appreciated that those skilled in the art, upon attaining an understanding of the foregoing, may readily conceive of alterations to, variations of, and equivalents to these aspects which fall within the spirit and scope of the present invention, which should be assessed accordingly to that of the appended claims.

1. A flexible wrap for supporting a portion of a body, the flexible wrap comprising:
an elastic band; and
an adhesive mounted on said elastic band near an end of said elastic band to secure said elastic band to a body, wherein the adhesive is adapted to be detachably mounted to the body.

2. The flexible wrap of claim 1 further comprising a releasable cover mounted on said adhesive, wherein said releasable cover is removed to expose said adhesive.

3. The flexible wrap of claim 1 wherein said adhesive is an adhesive layer that extends between lateral edges of said elastic band along said end of said elastic band.

4. The flexible wrap of claim 1 wherein said adhesive is adapted to be detachably mounted to skin on the body.

5. The flexible wrap of claim 1 wherein said adhesive is a hydrogel.

6. The flexible wrap of claim 1 wherein said elastic band includes a plurality of layers.

7. A flexible wrap for supporting a portion of a body, the flexible wrap comprising:
an elastic band; and
a plurality of fingers extending from an end of said elastic band, wherein said plurality of fingers are secured to an exposed section of said elastic band when the flexible wrap is attached to the body.

8. The flexible wrap of claim 7 further comprising a plurality of fasteners, wherein at least some of the fingers includes at least one fastener for securing said plurality of fingers to said exposed section of said elastic band.

9. The flexible wrap of claim 8 wherein each of the fingers includes at least one fastener for securing said plurality of fingers to said exposed section of said elastic band.

10. The flexible wrap of claim 7 wherein each of said plurality of fingers is integral with said elastic band.

11. The flexible wrap of claim 10 wherein said plurality of fingers includes at least one finger that extends from said end of said elastic band along a lateral edge of said elastic band and at least one other finger that extends from said end of said elastic band along an opposing lateral edge of said elastic band.

12. The flexible wrap of claim 11 wherein said plurality of fingers includes at least one finger that extends from a midsection of said end on said elastic band.

13. A flexible wrap for supporting a portion of a body, the flexible wrap comprising:
an elastic band having a first end and a second end;
an adhesive layer mounted on said elastic band near said first end to secure said elastic band to a body, wherein said adhesive layer is adapted to be detachably mounted to skin on the body;
a plurality of fingers extending from said second end of said elastic band, wherein said plurality of fingers are integral with said elastic band and secured to an exposed section of said elastic band when the flexible wrap is attached to the body; and
a plurality of fasteners, wherein each finger includes at least one fastener for securing said plurality of fingers to said exposed section of said elastic band.

14. The flexible wrap of claim 13 wherein said elastic band includes a plurality of layers.

15. A flexible wrap for supporting a portion of a body, the flexible wrap comprising:
an elastic band;
a pack that includes a midsection; and
a fastener extending along said midsection of said pack such that said fastener secures said pack to said elastic band.

16. The flexible wrap of claim 15 wherein said fastener is an adhesive.

17. The flexible wrap of claim 16 wherein said adhesive is an adhesive layer that extends between opposing edges of said pack.

18. The flexible wrap of claim 17 wherein said elastic band includes lateral edges and said adhesive layer is transverse to said lateral edges when said pack is secured to said elastic band.

19. The flexible wrap of claim 18 wherein said adhesive layer extends between said lateral edges of said elastic band when said pack is secured to said elastic band.

20. The flexible wrap of claim 16 wherein said adhesive is hydrogel

21. A method of supporting a portion of a body, the method comprising:
attaching an elastic band to the body using an adhesive; and
wrapping the elastic band around the portion of the body.

22. The method of claim 21 further comprising securing an end of the elastic band to an exposed section of the elastic band.

23. The method of claim 21 wherein attaching an elastic band to the body using an adhesive includes attaching an end of the elastic band to the body.

24. The method of claim 21 wherein attaching an elastic band to the body using an adhesive includes attaching the adhesive to skin on the body.

25. The method of claim 21 wherein attaching an elastic band to the body using an adhesive includes exposing the adhesive.

26. A method of supporting a portion of a body, the method comprising:
wrapping an elastic band around the portion of the body; and
securing a plurality of fingers that project from an end of the elastic band to an exposed section of the elastic band.

27. The method of claim 26 further comprising repositioning at least one of the plurality of fingers to customize pressure applied by the elastic band.

28. The method of claim 27 wherein repositioning at least one of the plurality of fingers includes disengaging the at least one of the plurality of fingers from the elastic band and then securing the at least one of the plurality of fingers to another part of the elastic band.

29. The method of claim 26 further comprising attaching the elastic band to the body.

30. The method of claim 29 wherein attaching the elastic band to the body includes attaching the elastic band to skin on the body using an adhesive.

31. The method of claim 26 wherein wrapping an elastic band around the portion of the body includes wrapping the elastic band around the body more than one time but less than two times.
